(19) United States
(12) Patent Application Publication
(10) Pub. No.: US 2014/0171672 A1
(43) Pub. Date: Jun. 19, 2014

(54) Method for Recovering Lipids from a Microorganism

(71) Applicants: Catherine LeGrand, Kalmar (SE); Martin Olofsson, Kalmar (SE)

(72) Inventors: Catherine LeGrand, Kalmar (SE); Martin Olofsson, Kalmar (SE)

(73) Assignee: NESTE OIL OYJ, Espoo (FI)

(21) Appl. No.: 14/007,890

(22) PCT Filed: Apr. 2, 2012
(86) PCT No.: PCT/FI2012/050332

§ 371 Date: Oct. 18, 2013

(30) Foreign Application Priority Data
Apr. 1, 2011 (EP) 11160790.9
Apr. 1, 2011 (US) 61470595

(51) Int. Cl. C12P 7/64; C12M 1/00

(52) U.S. Cl. CPC C12P 7/6463; C12M 47/06
USPC 554/204; 435/289.1

(57) ABSTRACT

A method is for recovering lipids from algae, which comprises providing a biomass of a lipid-producing algae. The cell wall and/or cell membrane of said algae is ruptured by algal cytotoxin thereby releasing lipid from the cell, and recovering said lipid. An integrated system is for recovering lipids from lipid-producing algae cells.

### FIELD OF THE INVENTION

The present invention relates to a method for recovering lipids from a lipid-producing microorganism.

The invention relates also to an integrated system for recovering lipids from lipid-producing algae cells.

### BACKGROUND

Microorganisms such as algae, bacteria and fungi may contain triglycerides up to 80% of their dry matter content. However, recovering lipids from biomass of microorganisms with conventional methods can encounter unexpected problems in regard to residual biomass since the used cell-breaking-method for microorganisms may affect negatively to the subsequent use of residual biomass in other applications.

For example some autotrophic algae are lipid-rich, robust and easy to cultivate. The difficulty with some algal species relates to their cell wall, which is practically impossible to break efficiently with conventional methods for releasing the lipids of the cells while at the same time keeping the quality of the residual algal biomass high enough for continued processing and utilization.

As a consequence of presently used conventional methods, based on chemical solvents and often high temperature or pressure, the residual microorganism biomass, such as algal cells, cannot be used to many high-value applications, e.g. functional proteins, because of denaturation of proteins, or food or feed, because of solvent residuals in the biomass. However, high-value applications are important in order to maximise the value chain of algae and to decrease price of the raw bio-oil. In addition, costs related to energy consumption (high temperature and pressure) and regeneration of large amount of solvents should be avoided.

US 2011/0076748 describes the use of an active ionic liquid to dissolve algae cell walls. The ionic liquid is used to dissolve and/or lyse an algae cell walls, which releases algae constituents used in the creation of energy, fuel, and/or cosmetic components. However, the method includes the use of heat and/or pressure.

There is thus still a need for a more gentle and energy-saving method for releasing and recovering lipids from algae.

### SUMMARY

The present invention tackles the above mentioned problems in a novel way by providing an alternative method for oil recovery from microbial biomass cells, especially from algae cells, based on solvent extraction, which is one of the major challenges in the applications using algal oil as feedstock for renewable diesel, such as NExBTL. Rupturing algae cells by means of unconventional biological means makes it possible to easily break of hard or otherwise unbreakable cell walls of microorganisms. This method can potentially be applied to several different types of algae cells and microbial cells. The biochemical means used in the above mentioned novel biochemical method will preferably break cell walls and/or cell membranes of lipid-producing microbial species in such a way that biochemical means will not themselves harm environment but instead will decompose when ended into nature.

The present invention is based on the idea to use the biochemical rupturing capacity of cytotoxic algae on cell membrane or cell wall of lipid rich algae or other microorganisms and thereafter the collection of lipid(s) from the mentioned lipid rich algae or other microorganisms by extracting oil droplets from water phase of algal or microbial aqueous slurry containing algal or microbial cell debris.

In a first aspect the present invention provides a method for recovering lipids from a lipid producing microorganism according to claim 1.

In a second aspect the present invention provides an integrated system for recovering lipids from lipid-producing algae cells according to claim 13.

### DETAILED DESCRIPTION OF THE INVENTION

The present invention provides new techniques for rupturing cells of lipid-producing microorganisms and subsequent collection of released intracellular lipids from the water phase.

In a first aspect the present invention provides a method for recovering lipids from a lipid-producing microorganism, which comprises the steps of
providing a biomass of a lipid-producing microorganism,
rupturing the cell wall and/or cell membrane of said microorganism by algal cytotoxin thereby releasing lipids from the microorganism cell, and
recovering said lipids.

By using algal cytotoxins in the above mentioned method, cell walls and/or cell membranes of lipid-producing algae or other microorganisms can be ruptured in such a way that it is possible to recover lipids without causing damage to the residual algal or microbial biomass. The proteins and other valuable components of the residual biomass can subsequently be used in other applications.

The method according to the current invention is a gentle and yet effective biological method for rupturing cell wall and/or cell membranes. The present method does not require the use of organic solvents or other chemicals that would have to be removed from the system before further use of the lipids. The method does not require the use of high energy in form of elevated temperature or high pressure. Therefore the method is cost-effective yet feasible in industrial scale. The algae producing the algal cytotoxins can be cultured in similar conditions compared to the lipid producing microorganisms and does not require special arrangements.

The method described above is especially suitable for rupturing cell walls of autotrophic algae belonging to different groups with semi-rigid or rigid wall (e.g. diatoms and certain green algae). The method is naturally applicable also for rupturing less rigid cell walls of algae or other microorganisms.

In a second aspect the invention provides an integrated system for recovering lipids from lipid-producing algae cells. The system comprises a growth vessel for lipid-producing algae and a growth vessel for a cytotoxin producing algae.

In the integrated system lipid-producing algae are cultivated under conditions suitable for producing lipids and said cytotoxin-producing algae are cultivated under conditions suitable for producing cytotoxins. Produced cytotoxins will be added to the growth vessel containing lipid-producing algae, in sufficient amount to rupture the cell membranes and/or cell walls of mentioned lipid-producing algae thereby releasing the lipid component from the cells. Thereafter lipids are recovered from the other cell components.

In an alternative embodiment of the above integrated system, lipid-producing algae and cytotoxin-producing algae are both cultivated in the same growth vessel and in such an environment that the cytotoxin production is suppressed. After the biomass of cytotoxin-producing algae is at a suitable level, the production of exotoxins is triggered for example by using suitable stress conditions.

As used herein, the definition “algal cytotoxin” refers to a toxic algal substance originating from algae, which toxic algal substance is able to rupture cell wall(s) and/or cell membrane(s) of algae or other microorganisms. These algal cytotoxins include for example extracellular algal toxins (exotoxins), excreted by microalgae. Herein the term “cytotoxin” is used to specify the cell wall or/and cell membrane rupturing action of an algal toxin released into the medium surrounding algal cells since algal toxins may also cause the death of algae with other mechanisms without actually rupturing the cell walls/cell membranes of said algae.

Toxic algal substances, as used herein, means same as algal toxins.

According to one embodiment of the invention algal cytotoxin(s) is/are selected so that it/they rupture(s) the cell wall and/or cell membrane of at least one microalgae selected from the group consisting of genera Phaeodactylum, Rhodomonas, Cryptomonas, Thalassiosira, Cyclotella, Haematococcus and Dunaliella.

According to one specific embodiment of the invention algal cytotoxin(s) are selected so that it/they rupture the cell wall/membrane of microalgae selected from the group consisting of Phaeodactylum and Rhodomonas.

Advantageously algal cytotoxin(s) is/are selected so that it/they rupture(s) the cell wall and/or cell membrane of a lipid-producing microalgae selected from the group consisting of Phaeodactylum spp.; and Rhodomonas spp., thereby releasing the lipid component from said algae cells. Phaeodactylum spp. belongs to diatoms, which have a very rigid cell wall. Since algal cytotoxins as shown here rupture the cell wall of Phaeodactylum spp., they potentially rupture the cell wall/membrane also of other diatoms, such as Cryptomonas spp., Thalassiosira spp. and Cyclotella spp. Rhodomonas spp. represents a typical algae genus, the members of which serve in many studies as model algae. Because algal cytotoxins have been shown here to rupture Rhodomonas spp., algal cytotoxins can potentially rupture microalgae having similar type or weaker cell wall as Rhodomonas spp. Such algae belong to genera consisting for example of Haematococcus spp.; and Dunaliella spp.

One of the main sources of algal cytotoxins are marine or freshwater plankton which comprises algae groups including diatoms, cyanobacteria, dinoflagellates, prymnesiophytes and raphidophytes, and which can produce potentially cytotoxic algal toxins into their surroundings. Whether all algal exotoxins really have the cytotoxic effect against certain algae can be tested separately in regard to the algae of interest and also at least one test microalgae as described above. If an exotoxin will rupture the cell wall and/or cell membrane of the test microalgae, it is likely that it will also rupture the cell wall/cell membrane of the lipid producing algae or other microorganism.

The algal cytotoxins, which can rupture or lyse the cell wall and/or cell membrane of algae, can be derived preferably from algae species belonging to the above mentioned groups: diatoms, cyanobacteria, dinoflagellates, prymnesiophytes or raphidophytes. The algae producing the algal cytotoxins should be easy to cultivate and the cytotoxin and its production stable over time.

The chemical structures of the algal cytotoxins can vary to some degree, since each alga may produce several different toxic algal substances. The mechanism by which each algal toxic substance acts on the algae and other microorganisms is generally not known. In case an algal toxic substance kills microorganisms, one common killing mechanism is the lysis of cell wall and/or cell membrane. The algal cytotoxins can usually be found among the algae, which excrete, in some conditions, extracellular toxins into the medium surrounding them.

Algal toxins, which have for example neurotoxic effects, are typically not excreted and are bound to the cells by which they are produced. Therefore, the harmful effects of some algal toxins can be avoided by using the cell free medium of algae, not containing cell-bound toxins.

In the following are given some examples of algal species belonging to the above mentioned algal genera whose algal toxins or toxic secondary metabolites may be cytotoxic, that is, they may be able to rupture cell membranes/cell walls of at least algae (including cyanobacteria), or other microorganisms including also cell wall and/or cell membrane of at least one of the following microalgae selected from the group consisting of genera Cryptomonas, Cyclotella, Dunaliella, Haematococcus, Phaeodactylum, Rhodomonas and Thalassiosira, preferably consisting of genera Phaeodactylum and Rhodomonas.

Potentially cytotoxic algae, which belong to cyanobacteria include the genera Anabaena, Aphanizomenon, Calothrix, Cylindrospermopsis, Fisherella, Gomphosphaeria, Hapalosiphon, Microcystis, Nodularia, and Nostoc. Examples of potentially cytotoxic algae belonging to dinoflagellates are Alexandrium, Coolia, Dinophysis, Heterocapsa, Karlodinium, Karenia, Ostreopsis, Peridinium and Prorocentrum. Examples of potentially cytotoxic algae belonging to prymnesiophytes are Chrysochromulina, Phaeocystis and Prymnesium. Examples of potentially cytotoxic diatoms are Pseudonitzschia and Nitzschia and examples of potentially cytotoxic raphidophytes are Heterosigma and Chattonella.

According to one embodiment of the invention the algal cytotoxin is produced by an algae species belonging to genus Alexandrium. It produces stable toxic algal substances, which can be used as cytotoxins, since they are able to rupture and lyse a whole range of algal cells, including for example those, belonging to flagellates and diatoms.

By algal cytotoxin(s) are meant one or more single cytotoxins or a composition of cytotoxins produced by an alga. The term algal cytotoxin encompasses here also chemical analogues or derivatives of algal cytotoxins.

By “algal cytotoxin” is here meant one algal cytotoxin or several algal cytotoxins.

The most preferred toxic algal substances for use as algal cytotoxins are those whose toxicity disappears fast from the water phase by chemical effect, such as temperature or light, or biological effect, such as bacterial degradation. These kind of algal cytotoxins are for example cytotoxins excreted by the genus Prymnesium, which is a fast-growing haptophyte. For example the algal cytotoxins produced by the mixotrophic P. parvum may be degraded by the effect of sunlight and UV-radiation.

In addition to algae, many algal toxins or toxic secondary metabolites of algae have also cytotoxic effect on cell membranes/cell walls of other microorganisms, such as bacteria, for example heterotrophic bacteria (Phycologia (2003) Vol 42 (4) 406-419). If these microorganisms can accumulate high intracellular lipid content, these lipids may be worth of recovering by using the method and means according to present invention.

According to another embodiment of the invention the algal cytotoxins encompass here toxic free fatty acids. Free fatty acids can be produced by algal cells and used according to the method of the invention. Also the chemical analogues or derivatives of free fatty acids can be used according to the invention.

A chemical analogue of an algal cytotoxin means a synthesized compound, which has substantially the same structure and effect to the microbial cell or cell membrane as the original algal cytotoxin.

As used herein the definition “rupturing” cells of algae or other microorganisms refers to a process which damages the cell walls and/or cell membranes of algae or other microorganisms by means of the action of an algal cytotoxin and which results in destruction, lysis, degradation, decomposition or loss of integrity of the cell walls/cell membranes in such an extent that it allows releasing of oil/lipids from the interior of mentioned algal or microbial cells.

As used herein the term “lipid” refers to a fatty substance, whose molecule generally contains, as a part, an aliphatic hydrocarbon chain, which dissolves in nonpolar organic solvents but is poorly soluble in water.

Lipids are an essential group of large molecules in living cells. Lipids comprise, for example, fats, oils, waxes, wax esters, sterols, terpenoids, isoprenoids, carotenoids, polyhydroxyalkanoates, fatty acids, fatty alcohols, fatty acid esters, phospholipids, glycolipids, sphingolipids and acylglycerols, such as monoglycerides (monoacylglycerol), diglycerides (diacylglycerol) or triglycerides (triacylglycerol, TAG). In the present invention desired lipids to be recovered in the product include fats, oils, waxes and fatty acids and their derivatives.

As used here the term “biomass” is meant biomass derived from a culture containing microorganisms including bacteria, cyanobacteria, fungi such as yeasts, filamentous fungi and moulds, archaea, protists; microscopic plants such as algae, microalgae or plankton, preferably bacteria, cyanobacteria, archaea, protists; microscopic plants, such as algae, microalgae or plankton. This term includes also a ready-made, frozen or otherwise previously worked biomass, which is subsequently used in this method.

Definition “providing a biomass” comprises herein the use of a biomass derived from a culture of algae or other microorganisms or the use of a ready-made frozen or otherwise previously worked biomass.

Most lipid-producing microorganisms are unicellular i.e. single-celled, however, some microscopic multicellular organisms are also able to accumulate lipids. The microorganisms readily accumulate lipids or have been genetically modified to accumulate lipids or to improve accumulation of lipids. In a preferred embodiment of the present invention lipid containing microbial biomass is selected from the group of bacteria, cyanobacteria, archaea, protists and microalgae, more preferably from the group of algae, microalgae and cyanobacteria.

Preferably, suitable microalgae comprise one or more representatives from the following taxonomic classes: Chlorophyceae, Cryptophyceae (recoiling algae), Chrysophyceae, Diatomophyceae (diatoms), Dinophyceae (dinoflagellates), Euglenophyceae, Eustigmatophyceae, Pavlovophyceae, Pedinophyceae, Prasinophyceae, Prymnesiophyceae (haptophyte algae) and Raphidophyceae.

In a preferred embodiment the microbial biomass comprises freshwater and marine microalgae genera comprising Achnantes, Agmenellum, Amphiprora, Amphora, Anabaena, Anabaenopsis, Ankistrodesmus, Arthrospira, Attheya, Boeklovia, Botryococcus, Biddulphia, Brachiomonas, Bracteococcus, Carteria, Chaetoceros, Characium, Chlamydomonas, Cricophaera, Crypthecodinium, Cryptomonas, Chlorella, Chlorococcum, Chrysophaera, Coccochloris, Cocconeis, Cyclotella, Cylindrotheca, Dermocarpa, Dunaliella, Ellipsoidon, Entomoneis, Euglena, Eremosphaera, Extubocellulus, Franceia, Fragilaria, Gleocapsa, Gleothamnion, Hantzschia, Haematococcus, Hormotilopsis, Hymenomonas, Isochrysis, Lepocinclis, Melosira, Minidiscus, Micractinum, Microcystis, Monallanthus, Monoraphidium, Muriellopsis, Nannochloris, Nannochloropsis, Navicula, Neochloris, Nephroselmis, Nitzschia, Nodularia, Nostoc, Ochromonas, Oedogonium, Oocystis, Oscillatoria, Papiliocellulus, Parachlorella, Pascheria, Pavlova, Peridinium, Phaeodactylum, Phagus, Plankthothrix, Platymonas, Plectonema, Pleurochrysis, Phormidium, Pleurosigma, Porphyridium, Prymnesium, Pseudochlorella, Pyramimonas, Pyrobotrus, Radiosphaera, Rhodomonas, Rhodosorus, Sarcinoid, Scenedesmus, Schizochytrium, Scrippsiella, Seminavis, Skeletonema, Spirogyra, Spirulina, Stichococcus, Synechococcus, Synechocystis, Synedra, Tetraedron, Thalassiosira, Trachyneis, Traustrochytrium, Trentepholia, Ulkenia, Viridiella, Volvox and Xenococcus, preferably Rhodomonas, Phaeodactylum spp., such as P. tricornutum and Dunaliella, such as D. marina, D. saline, or D. tertiolecta, or bacteria selected from the group consisting of the genera Acinetobacter, Actinobacter, Aerogenes, Alcanivorax, Arthrobacter, Bacillus, Clostridium, Cupriviadus, Dietzia, Gordonia, Escherichia, Flexibacterium, Micrococcus, Mycobacterium, Nocardia, Pseudomonas, Ralstonia, Rhodococcus, Rhodomicrobium, Rhodopseudomonas, Shewanella, Shigella, Streptomyces, Wautersia and Vibrio, preferably Rhodococcus opacus, Acinetobacter, Nocardia or Streptomyces.

By the term such as “advantageous”, “preferred”, “preferential” or “special” is meant herein that the selected subject matter is advantageously but not necessarily used in this connection because there is some advantages relating to its use. However there can be also some disadvantages involving to the use of subject matter that is “advantageous”, “preferred”, “preferential” or “special” and therefore these terms should not be interpreted in any restrictive way.

General Method

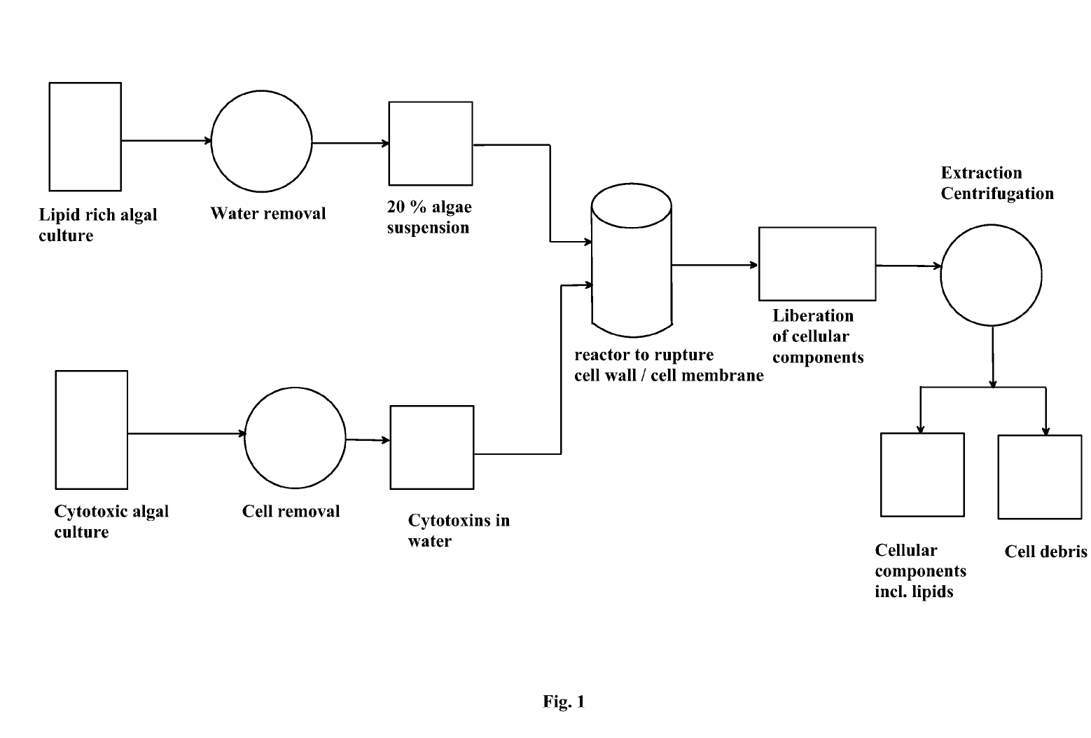

The general method used for recovering products such as lipids from microbial biomass, especially from algae cells is outlined in FIG. 1.

Although this method is described for recovering lipids from microalgae the same kind of method can be used also for recovering lipids or other products from suitable microbial biomasses as far as their cell walls and/or cell membranes are breakable or rupturable by toxic algal substances.

The conditions used in the method as described herein can be used also when testing whether an algal cytotoxin ruptures the cell wall and or membrane of algae or other microorganisms.

Typically lipid-producing algae are cultivated at a temperature of 4 to 50° C. The pH is typically adjusted to pH 5 to 11.

The ratio of algal cytotoxin(s) to microorganism/algae cell can be adjusted to range of dose: target ratio (cell:cell) 1:100 000 to 1:10, preferably 1:1000 to 1:100.

The cytotoxin(s) is/are typically incubated with the biomass 2 to 24 hours, preferably from 3 to 12 hours.

Algal cytotoxins may be used in the form of cultivation medium of the cytotoxic algae or in the form of cell-free suspension of the cultivation medium, preferably as a cell-free suspension of the cultivation medium.

The lipid-producing algae can be cultivated under cultivation conditions comprising a stress induction phase, such as nutrient deprivation, pH variations or excess of oxygen species, causing increased lipid production.

These toxic algal substances will break or rupture microalgae selected from the group consisting at least one of Phaeodactylum spp.; and Rhodomonas spp.; Cryptomonas spp.; Cyclotella spp.; Dunaliella spp.; Haematococcus spp.; and Thalassiosira spp., preferably selected from the group consisting at least one of Phaeodactylum spp.; and Rhodomonas spp. Microalgae belonging to these genera and species can be used when testing whether an algal substance(s) is suitable for use in the method or system according to this invention.

Other suitable microbial or algal biomasses have been listed above when discussing the meaning of definition “biomass”.

Algae can grow either in light conditions or dark conditions. In light conditions algal growth is photoautotrophic or autotrophic and the light energy is required for growth. In dark conditions the algal growth is heterotrophic which means, that other kind of energy than light is required for growth. Lipid-producing algae can be cultivated in both of these conditions. However autotrophic growth and heterotrophic growth requires different growth conditions and nutrients, which should be taken into account when considering cultivation.

During the heterotrophic and autotrophic growth certain growth conditions and nutrients can be used for promoting oil production inside the algae cells. Additionally the oil production of some lipid-producing algae can be enhanced by cultivating lipid-producing algae under cultivation conditions comprising a stress induction phase. Possibilities for causing stress induction phase are for example, light deprivation, injection of reactive oxygen, pH or nutrition changes or chemical addition. For example by changing the proportion of nitrogen to phosphorus during the growth phase of algae can boost remarkable lipid production as demonstrated also in Examples 1-6 below.

The microbial biomass to be processed may be obtained directly from cultivation or growth system, such as from a growth vessel. When the method presented in FIG. 1 is used for recovering oil from lipid-producing algae, the method is usually commenced by separately culturing lipid-producing algae and a cytotoxin-producing algae in different growth vessels.

Alternatively the method presented in FIG. 1 can be modified so, that lipid-producing algae and toxin-producing algae are cultivated in the same growth vessel. However, the culturing conditions are such, that the extracellular toxin production is suppressed. After the biomass of toxin-producing algae is at a suitable level, the production of exotoxins is triggered by changing culturing conditions, for example by subjecting toxin-producing algae to suitable stress induction phase.

Growth vessel as used herein means a closed solar photoreactor or a closed, artificially illuminated photoreactor, an open container or raceway, or a reservoir or a natural or artificial water pond. Reservoir or water pond may contain natural fresh water or natural seawater or artificial seawater.

Each growth vessel may include a carbon dioxide source or carbon dioxide can be added into the growth vessel during the cultivation for adjusting pH. Growth environment can also contain suitable temperature controlling, aerating and circulating means. It may also be necessary to supply nutrients and modify nutrient content to each other. If algae are cultivated in a closed photoreactor, the reactor should be provided with illumination.

The lipid-producing algae to be cultured in a growth vessel may comprise a single algal species or a mixed combination of two or more algal species.

Wide variety of algae can be used for production of biomass, from which lipid or oil can be recovered. The method is suitable for autotrophic, heterotrophic or mixotrophic algae. Most common lipid-producing algae can be found from the groups of diatoms, chlorophytes (green algae), cyanobacteria (blue-green algae), chrysophytes (golden-brown algae), dinoflagellates and haptophytes. Suitable lipid-producing algae can be found among those algae mentioned above or from discussion relating to the term “biomass”.

Microbial biomass to be processed is treated by generally known methods, such as centrifugation, filtration, decanting, floatation or sedimentation possible assisted by flocculation and water evaporation to remove excess water or aqueous growth solution. Microalgae, bacteria, archaea biomass is preferably filtered or centrifuged before processing. On the other hand, biomass from solid state cultivation, immobilized cultivation or the like may be used by slurring it into aqueous media, if necessary.

By the term “wet” is meant algal biomass which originates from aqueous cultivation solution and from which excess water is removed by common low energy consuming water removal processes, such as filtering or the like and which is not specifically dried. Alternatively, solid dry algal biomass may be slurried into an aqueous form.

In the process described in FIG. 1, the lipid-producing algae are first cultivated in a first growth vessel and the cultivated algae cells are then partially dried by removing excess water by evaporation, sedimentation, centrifugation, assisted possibly with flocculation.

The algal cytotoxins used in this method can be produced by a suitable algae mentioned above when discussing the definition “algal cytotoxin”. Preferably algal cytotoxins are produced by a single phytoplankton species naturally present in marine or freshwater environment or by the mixture of two or more phytoplankton species.

The cytotoxin-producing algae are cultivated in a second growth vessel (that is, in a closed photoreactor, open container or a reservoir or a natural or artificial water pond). The growth conditions and nutrients should be adjusted according to requirements of algal species to be grown. Algal cytotoxins producing algae comprise different species with different requirements as to their growth conditions, nutrients and nutrient ratios. For example cytotoxin-producing dinoflagellates include species, ranging from obligate autotrophs to mixotrophs and therefore there exists a wide variety of factors, which affect their toxicity and growth. For example pH, temperature, salinity of growth medium and nutrient limitations can affect the toxicity of algal cytotoxins. After cytotoxin-producing algae have been cultivated in a growth vessel, the algal slurry is filtered for removing algal cells and recovering cell-free filtrate, which contains the algal cytotoxins.

The filtrate, which contains algal cytotoxins, is then added into partially dried algal biomass. In one embodiment the algal cytotoxins and water containing filtrate is added to the dried algal biomass containing algae cells in such an amount, that the proportion of algal cytotoxins producing algae cells, from which the algal toxins have been recovered, to lipid-producing algae cells will be from 1:100 000 to 1:10 preferably from 1:1000 to 1:100 (cell/cell). This virtual cell-proportion depends on the quality and quantity of cytotoxins present in the filtrate and the toxicity of cytotoxins against lipid-producing algae cells. After cytotoxin(s) containing aqueous filtrate has been added into partially dried algae cells the solids content of this aqueous algal suspension is preferable about 20 wt-% the rest of suspension being mostly water.

Alternatively the cytotoxin-producing algal culture is added without filtration or extraction to partially dried lipid-rich algal cells. It is possible also to combine the lipid-producing algal culture to the cytotoxin-producing algal culture without first processing neither of these algal cultures and thereafter to recover the lipids from this combined algal suspension.

After algal cytotoxins have been added into the lipid-rich algal culture, cytotoxic substances will affect to the cell walls/cell membranes of the algae cells by rupturing them. Usually the cell walls/cell membranes of algae lyse completely due to effect of cytotoxin(s) and lipids will be released into slurry of algal debris, which includes lipids, cell wall debris, intracellular products, enzymes, by-products etc.

The lipids can be removed from mentioned aqueous slurry of algal debris by conventional methods, such as extraction, centrifugation and/or filtering, for example by means of an extraction column.

The algal debris (biomass), which has been left after the removal of lipids, is in a good condition and can be utilized in subsequent reprocessing stages for producing of other valuable products. The recovered lipids can be used in the production of biodiesel, renewable diesel, jet fuel, gasoline or base oil components.

The method can be used also for recovering oil lipids from the microorganisms, such as microalgae or bacteria mentioned above. Especially suitable this method is for recovering oil from following algal species: Haematococcus spp.; Dunaliella spp. such as Dunaliella tertiolecta, D. marina, D. saline (green algae), Phaeodactylum spp. such as Phaeodactylum tricornutum (diatom), Thalassiosira spp. such as T. pseudonana, T. weissflogii and Cyclotella spp. (diatom); Cryptomonas spp. and Rhodomonas spp. such as Rhodomonas saline and Rhodomonas lacustris (recoiling algae). These microalgae are capable of accumulating high lipid content.

The invention is now described in more detail by means of examples.

### EXAMPLES

Lipid-Producing Algae

Chlorophyte Dunaliella tertiolecta (CCMP 1320)

Diatom Phaeodactylum tricornatum (CCMP 2928)

Cryptophyta Rhodomonas saline (KAC 30)

Cytotoxin-Producing Algae

Preferable the used cytotoxin is degradable in the environment by chemical or biological effect such as temperature, sunlight (or artificial UV-light), or bacteria, or binding to organic matrices. However, for the purpose of illustrating the validity of our method in rupturing the cell walls of lipid-producing algae and subsequent lipid recovery from aqueous algal cell debris we used here more stable algal cytotoxins originating from the dinoflagellate genus Alexandrium. These cytotoxins were produced by Alexandrium tamarense during exponential growth in a photoreactor supplied with an artificial illumination. During the cultivation of lipid-producing algae was introduced a stress inducing phase by restricting the access to nutrients during late growth phase.

### Examples 1 and 2

Culturing of Lipid-Producing Algae and Potentially Cytotoxic Algae

Two oil-producing algae, the chlorophyte Dunaliella tertiolecta (CCMP 1320) and the diatom Phaeodactylum tricornatum (CCMP 2928) were cultivated in f/2 medium (N:P=24) prepared from filtered artificial seawater (35%) in aerated batch cultures (2-10 L). Oil-producing algae were grown under controlled conditions at 20° C. on a 16:8 h light-dark cycle a under cool white fluorescent light at an irradiance of 220 μE m-² s-¹. CO₂ was injected daily to lower the pH from 9-9.5 to 8-8.5, and grown until exponential phase at a biomass of 3×10⁵-5×10⁶ cells mL⁻¹ containing 10-20% lipids (dry w/dry w).

To boost lipid accumulation in the cells, a stress induction phase was commenced by inducing nutrient stress in the oil-producing algal cultures through harvesting 20-30% of the culture volume before replenishing the culture with modified f/2 (N:P=2). Lipid content reached 40-50% cellular lipid (dry w/dry w) over a period of 1-2 days.

Quantification of lipid content was carried out using a modified Bligh & Dyer (1959) method.

Potentially cytotoxic algal substances producing Alexandrium tamarense was grown in K-medium (N:P=24) prepared from filtered seawater (salinity 32) in 2 L batch cultures under controlled conditions at 15° C. on a 16:8 h light-dark cycle under cool white fluorescent light at an irradiance of 65 μE m⁻² s-⁻¹.

### Examples 3 and 4

Lipid Rich Algae were Harvested Through Centrifugation (20 min., 100×g)

Algal toxic substances from A. tamarense (Tillmann & John, 2002) were released from the cells to the surrounding medium. To collect algal toxic substances, cell-free filtrate of A. tamarense was prepared from dense stationary phase cultures (8-20×10³ cells mL⁻¹) by gently filtering the culture through a 10 μm mesh nylon net.

### Example 5

Partial Drying of the Algal Cells

Lipid rich algal wet biomass was obtained after centrifugation (40 mL) of the target cultures and re-suspension (Dunaliella: 0.2-1.4×10⁵ cells mL or 4.5-5.8 g L⁻ dry weight; Phaeodactylum: 2.2-4.5×10⁸ cells mL⁻¹). The supernatant was discarded except for 3-4 ml that was retained in the centrifugation tube and used for resuspension of the pelleted biomass. Re-suspension was carried out in order to obtain a practical suspension easy to work with.

### Example 6

Cell Membrane Rupture

Lipid rich algae cell membrane was biologically ruptured through the addition of toxic algal substances originating to A. tamarense (cell-free filtrate from examples 3 and 4). A combination of dose response assays and time series were carried out to define the optimal parameters (dose:target ratio and exposure time) resulting in the rupture of the cell membrane of the target (Dunaliella, Phaeodactylum). Algal cytotoxins were added to target cells in the range of dose:target ratio (cell:cell) of 1:1000 to 1:100, and incubated over 24 h with sampling every 15 minutes over 4 h, and a final sampling at 24 h. After the addition of algal cytotoxins to target cells, these cells were immediately stained with the fluorochrome Nile Red (3.9 μM final concentration) to stain the cellular lipid droplets (Nile Red: A selective fluorescent stain for intracellular lipid droplets, Greenspan et al., The Journal of Cell Biology, Vol 100, 965-973, March 1985). Stained cells were stored 10 min in the dark prior to observation of the cells in epifluorescence microscopy (blue light). The process of lipid release from the cellular matrix was followed using 15 min intervals over 4 hours. After short exposure (1-4 h) to algal cytotoxins, lipid-rich algae cell lysis occurs and cells break open thus confirming cytotoxic effect of mentioned toxic algal substances. Microscopic observation revealed that within 1 h of exposure to these algal cytotoxins, the donor cell membrane started to rupture in both Dunaliella and Phaeodactylum. After rupture of the cell membrane (3-4 h), oil droplets were released from the cell matrix into the surrounding water. Lipid release can be uneven in different cells; oil droplets could still be observed in the cellular matrix after 4 h. This process was observed using epifluorescence microscopy (40-100×, blue light at 488 nm). Oil droplets when stained with the fluorochrome Nile Red, were seen as bright yellow while the rest of the algae cell was red.

### Example 7

Lipid Producing Microalgae

Rhodomonas saline (KAC 3) was cultivated in f/2 medium (N:P=24) prepared from filtered Baltic seawater with NaCl in batch culture (1 L). The algae was grown under controlled conditions at 20° C. on a 16:8 h light-dark cycle under cool white fluorescent light at an irradiance of 200 μE m-² s-¹. The culture was grown to late exponential phase at a biomass of 1-3×10⁶ cells mL⁻¹.

To boost lipid accumulation in the cells, a stress induction phase was commenced by inducing nutrient stress in the oil-producing algal cultures through harvesting 20-30% of the culture volume before replenishing the culture with modified f/2 (N:P=2). Experiments were conducted 2-3 days after stress induction.

The cell membrane of Rhodomonas was biologically ruptured through the addition of toxic algal substances originating to A. tamarense. Before the addition of cytotoxins Rhodomonas cells were stained with the fluorochrome Nile Red (3.9 μM final concentration) to stain the cellular lipid droplets (Nile Red: A selective fluorescent stain for intracellular lipid droplets, Greenspan et al., The Journal of Cell Biology, Vol 100, 965-973, March 1985). Algal cytotoxins were added to target cells in dose:target ratio (cell:cell) of 1:5-1:20. Samples of Rhodomonas cells were counted in the epifluorescence microscope (approximately 300 cells in15 or 60 μL) 15-45 min after incubation with cytotoxins. By observation, all cells counted in the microscope were clearly ruptured. 62% of total cells and 82% of cells stained with Nile Red (NR) were releasing cellular lipid droplets (Table 1).

TABLE 1
The effect of cytotoxins on different species of algae and the efficiency of lipid release
from these lipid-rich algae after incubation with cytotoxins. In terms of dead cells
and efficiency of release approximately 300 cells were counted in 15 or 60 μL.

| | | | | % of | % of NR | % of |
|---|---|---|---|---|---|---|
| | | Target | | total cells | stained cells | dead cells |
| | Cell conc. | dose | Dead | releasing | releasing | releasing |
| | (cells/ml) | ratio | cells % | lipids | lipids | lipids |
| Rhodomonas | 0.65 × 10⁵-2.0 × 10⁵ | 1:5-1:20 | 95 within | 82 | 82 | 62 |
| | | | 1 h | | | |
| Dunaliella | 0.4 × 10⁶-1.0 × 10⁶ | 1:30-1:100 | 95 within | 47 | 53 | 49 |
| | | | 1 h | | | |
| Phaeodactylum | 0.4 × 10⁶-1.0 × 10⁶ | 1:30-1:100 | 95 after | <5 | <5 | <5 |
| | | | 20 h | | | |

1. A method for recovering lipids from a lipid-producing microorganism, which comprises:
providing biomass of a lipid-producing microorganism,
rupturing the cell wall and/or cell membrane of said microorganism by algal cytotoxin thereby releasing lipids from the microorganism cell, and
recovering said lipids.

2. The method according to claim 1, wherein the microorganism is a lipid-producing algae.

3. The method according to claim 1, wherein the algal cytotoxin is selected so that it ruptures the cell wall and/or cell membrane of a microalgae selected from the group consisting of the microalgae selected from the group consisting of genera Phaeodactylum, Rhodomonas, Cryptomonas, Thalassiosira, Cyclotella, Haematococcus and Dunaliella, preferably of genera Phaeodactylum and Rhodomonas.

4. The method according to claim 1, wherein the cytotoxin producing algae is selected from the group of cyanobacteria, diatoms, dinoflagellates, prymnesiophytes and raphidophytes, preferably from the group of genera Anabaena, Aphanizomenon, Calothrix, Cylindrospermopsis, Fisherella, Gomphosphaeria, Hapalosiphon, Microcystis, Nodularia, Nostoc, Alexandrium, Coolia, Dinophysis, Heterocapsa, Karlodinium, Karenia, Ostreopsis, Peridinium, Prorocentrum, Chrysochromulina, Phaeocystis, Prymnesium, Pseudonitzschia, Nitzschia, Heterosigma and Chattonella.

5. The method according to claim 1, wherein the cytotoxin producing algae is selected from the genus Alexandrium, most preferably from species Alexandrium tamarense.

6. The method according to claim 1, wherein the algal cytotoxin is free fatty acids.

7. The method according to claim 1, wherein the lipids are recovered by extraction and/or centrifugation.

8. The method according to claim 2, wherein the lipid-producing algae cell is produced by:
harvesting the lipid-producing algae cells from the algae culture, and
drying the algae cells to a water content of less than 80 w-%.

9. The method according to claim 1, wherein the microorganism or algae cells are dried by at least one of evaporation, flocculation and centrifugation.

10. The method according to claim 1, wherein said algal cytotoxin is in the form of cell-free suspension of the cultivation medium of cytotoxic algae.

11. The method according to claim 1, wherein the cytotoxin is incubated with the biomass 2 to 24 hours, preferably from 3 to 12 hours.

12. The method according to claim 1, wherein the lipid-producing algae is selected from the group of Chlorophyceae (green algae), Cryptophyceae (recoiling algae), Chrysophyceae (golden brown algae), Diatomophyceae (diatoms), Dinophyceae (dinoflagellates), Euglenophyceae, Eustigmatophyceae, Pavlovophyceae, Pedinophyceae, Prasinophyceae, Prymnesiophyceae (haptophyte algae) or Raphidophyceae.

13. An integrated system for recovering lipids from lipid-producing algae cells, comprising:
a first growth vessel for lipid-producing algae, and a second growth vessel for cytotoxin producing algae,
wherein said lipid-producing algae is cultivated under conditions suitable for lipid production and said cytotoxin producing algae is cultivated under conditions suitable for cytotoxin production, said cytotoxins being added from second growth vessel comprising cytotoxin producing algae to the first growth vessel comprising lipid-producing algae in sufficient amount to rupture at least one of the cell walls and cell membranes of the lipid-producing algae thereby releasing the lipid component from the cell, and recovering said lipids from the other cell components.

14. The system according to claim 13, wherein the cytotoxin is in the form of cell free suspension of the cultivation medium of the cytotoxin producing algae.
